(19) United States
(12) Patent Application Publication
(10) Pub. No.: US 2020/0276145 A1
(43) Pub. Date: Sep. 3, 2020

(54) REDUCING SIDE EFFECTS OF SHORT ACTING NO DONORS

(71) Applicant: G. POHL-BOSKAMP GMBH & CO. KG, Hohenlockstedt (DE)

(72) Inventor: Marianne BOSKAMP, Bekmünde (DE)

(21) Appl. No.: 16/467,883

(22) PCT Filed: Dec. 14, 2017
(86) PCT No.: PCT/EP2017/082932

§ 371 Date: Jun. 7, 2019

(30) Foreign Application Priority Data
Dec. 14, 2016 (EP) 16204170.1

(51) Int. Cl. A61K 31/21; A61K 9/00; A61P 9/10; A61P 43/00

(52) U.S. Cl. CPC A61K 31/21; A61P 43/00; A61P 9/10; A61K 9/006

(57) ABSTRACT

The present invention pertains to a short acting NO donor for use in a method of reducing side effects of a treatment with said short acting NO donor, comprising administering during a defined time period a gradually increasing amount of said short acting NO donor in an intermitting manner. The present invention is further directed to a short acting NO donor for use in a method for the prevention or treatment of an arterial insufficiency, wherein the NO donor is administered in an intermitting manner, and wherein during the initial phase of the administration, the amount of said short acting NO donor is gradually increased.

### RELATED APPLICATIONS

This application is a 35 U.S.C. § 371 filing of International Patent Application No. PCT/EP2017/082932, filed Dec. 14, 2017, which claims priority to European Patent Application No. 16204170.1, filed Dec. 14, 2016, the entire disclosures of which are hereby incorporated herein by reference.

The present invention relates to methods for reducing side effects of a treatment with a short acting NO (nitric oxide) donor.

Short acting NO donors are a well-known treatment of different diseases having their most frequent use in the cardiovascular field.

Cardiovascular diseases as well as other diseases involving a cardiovascular and, more specifically, arterial insufficiency affect a rising patient population, head international mortality and morbidity statistics and have an enormous economic importance. In Germany, for example, about 280.000 patients suffer every year from a cardiac infarct, while about 65.000 patients die.

One important reason for a cardiovascular disease is the partial or complete occlusion of arterial vessels resulting in a reduced supply of oxygen and nutrients of the tissue supplied by the arterial vessel.

Angina pectoris, the medical term for chest pain or discomfort, is a clinical syndrome reflecting inadequate oxygen supply for myocardial metabolic demands with resultant ischemia and is generally caused by obstruction (stenosis), spasm, endothelial or microvascular dysfunction of coronary arteries. Thus, angina pectoris is a symptom of an underlying heart problem, usually coronary artery disease (CAD).

(Short acting) NO donors, and especially nitroglycerin (glyceryl trinitrate, GTN), are used since decades as vasodilating agents in the above mentioned cardiovascular setting for the symptomatic treatment of cardiovascular diseases like coronary artery disease (also referred to as ischemic heart disease (IHD) or coronary heart disease (CHD)), which is the leading cause of death and disability worldwide (McGrae McDermott M., Journal of the American Medical Association, 2007, 297 (11): 1253-1255).

NO donors have been and still are used to treat the symptoms of these diseases without addressing the basic cause, i.e. the etiology, of the underlying disease due to their vasodilating effect on veins and arteries, resulting in a reduced workload and energy consumption of the heart (by decreasing preload and afterload) as well as an increased myocardial oxygen supply (by dilating the coronary arteries). These symptoms include chest pain, pressure, discomfort, or dyspnea.

Due to its short half-life time in blood plasma nitroglycerin is regarded as a short acting NO donor. Short acting NO donors, especially nitroglycerin, have been and are primarily used for the acute relief or acute prophylaxis of angina pectoris attacks, the most common symptom of CAD (Fox K. et al., Guidelines on the management of stable angina pectoris: full text. The Task Force on the Management of Stable Angina Pectoris of the European Society of Cardiology. European Heart Journal doi:10.1093/eurheartj/eh1002; Gibbons R. J. et al., ACC/AHA/ACP-ASIM Guidelines for the Management of Patients With Chronic Stable Angina. Journal of the American College of Cardiology, 1999, 33 (7): 2092-2197).

Unfortunately, the medical use of the pharmaceutically important NO donors produces frequent side effects in a dose-related manner. This is very well known for the use of the NO donors and organic nitrates nitroglycerin and isosorbide dinitrate (ISDN). They may cause side effects like nitrate headache, blood pressure drop, nausea. When used as a rescue medication in case of an acute ischemic event like, for example, an angina pectoris attack due to coronary artery disease, the side effects are tolerated by most of the patients due to the threatening situation of the acute event.

When used as an acute rescue medication, short acting NO donors are administered in a way that they show a rapid onset and short duration of action with regard to the duration of their pharmacological effect, i.e. their vasodilating action. Examples are intravenously administered GTN or sublingually administered GTN or ISDN formulated in immediate-release dosage forms. When administered in this manner both NO donors reach their peak blood plasma levels within minutes after the administration resulting in the fast onset of action.

When NO donors are used prophylactically as a long-term treatment, i.e. for the long-term prophylaxis of angina pectoris attacks, the side effects are known to be most prominent at the beginning of the therapy but subside during a period of days to weeks when the patient ('s) (body) gets used to the treatment.

For such long-term NO treatment, the NO donors have to be administered in a way that that elevated plasma or tissue levels of the NO donor in a therapeutically sufficient amount are generated in the subject resulting in a long duration of vasodilative action. To keep NO donor levels for a long period of time on a high, elevated level in the subject, short acting NO donors have either to be administered continuously (e.g. by continuous infusion of GTN) or by using an extended-release dosage form (e.g. such as a transdermal GTN patch with a controlled release of GTN from a reservoir) or oral retard preparations (e.g. oral ISDN retard). Alternatively, the patient is to be switched to long acting NO donors (e.g. oral isosorbide mononitrate (ISMN)) that have a long half-life time in the subject's body after their administration and are therefore long acting resulting in a prolonged antianginal activity.

The halftime of isosorbide mononitrate after oral administration is around 5 hours.

In WO 2013/178715, it is disclosed that short acting NO donors are able to treat and/or to prevent arterial insufficiencies via the induction of arteriogenesis when administered in an intermitting manner.

This implicates that short acting donors are used in a new and different way other than in the emergency setting described so far. In this case, patients are not in a threatening situation and will therefore more likely refuse to accept additional side effects caused by such a therapy.

Arteriogenesis is a process in which already pre-existing small arteriolar collaterals can develop to full functional conductance arteries which bypass the site of an arterial occlusion and/or compensate blood flow to ischemic territories supplied by the insufficient artery. Consequently, arteriogenesis is a highly effective endogenous mechanism for the maintenance and regeneration of the blood flow after an acute or chronic occlusive event in an arterial vessel. In this case, the collaterals can function as natural bypasses.

Arteriogenesis is a process distinct from angiogenesis or neovascularization, where a de-novo formation of arterial vessels occur (Buschmann I. and Schaper W., Journal of Pathology, 2000, 190: 338-342).

Thus, in contrast to the acute symptomatic treatment of cardiovascular diseases due to the vasodilating effect, short acting NO donors can be used intermittently to specifically treat or prevent diseases, i.e. arterial insufficiencies, via the induction of arteriogenesis. The latter addresses the basic cause of the disease, i.e. the insufficient blood flow to ischemic territories supplied by the insufficient artery, by means of augmentation of collateral blood flow.

Consequently, short acting NO donors represent useful medicaments for both the symptomatic treatment of cardiovascular diseases in an emergency setting, i.e. as a rescue medication for the short-term relief or prophylaxis of acute symptoms of the diseases, as well as for the long term prevention or treatment of arterial insufficiencies, such as e.g. cardiovascular diseases.

However, a treatment with short acting NO donors, as described above, is often impaired by the fact that their administration is accompanied by diverse side effects which may severely influence the compliance of the patients.

During the therapy with short acting NO donors such as nitroglycerin or ISDN, the following side effects have been monitored: headache is a very common (10% or more) side effect which is reported in up to 64% of the cases when short acting NO donors are administered for therapeutical means. Common (1% to 10%) side effects are dizziness, lightheadedness, syncope, vertigo and drowsiness. Rarely (0.1% to 0.01%) seen side effects are severe and prolonged headache. Very rare side effects (less than 0.01%) are in form of cerebral ischemia, other side effects that have been reported but without a particular frequency are faintness and somnolence.

Side effects can occur more frequently or severely in special populations. For example, hypotension or dizziness are more likely to occur in small, lightweight persons. Also, populations with different comorbidities may show a higher frequency of side effects.

The above mentioned side effects are known to negatively influence the patient's compliance especially in a situation which is not a medical emergency. When short acting NO donors are administered in an intermittent manner, e.g. for the induction of arteriogenesis, the side effects will therefore refrain patients from using short acting NO donors or will result in a reduced or insufficient compliance.

In view of the above, there is a need for methods for reducing the side effects of short acting NO donors especially when these NO donors are used in form of a long term intermittent treatment in a non-emergency setting.

In the context of the present invention, and as outlined in the examples, it has surprisingly been shown that the administration of a gradually increasing amount of a short acting NO donor in an intermitting manner results in the reduction of side effects.

The intermittent administration of short acting NO donors, as referred to in the context of the present invention, is used to create repetitive short-lasting episodes of high NO donor or NO blood plasma or tissue levels. On the one hand, the intermittent administration of short acting NO donors has the improved effect that it mimics the physiological situation of the organism as, for example, comparable to the endogenous release of NO upon physical training. On the other hand, the intermittent administration of a short acting NO donor avoids that the subject is developing tolerances against the NO donor and that the subject is developing endothelial dysfunction. The induction of endothelial dysfunction is a parameter which has a prognostic significance in patients with coronary artery disease. The development of tolerances and the induction of endothelial dysfunction are well known disadvantages caused by the sustained, long term exposure to NO donors (Uxa A. et al., Journal of Cardiovascular Pharmacology, 2010, 56 (4): 354-359).

In a first aspect, the present invention relates to a short acting NO donor for use in a method of reducing side effects of a treatment with said short acting NO donor, comprising administering during a defined time period a gradually increasing amount of said short acting NO donor in an intermitting manner.

Equally, the invention relates to a method of reducing side effects of a treatment with a short acting NO donor, wherein the method comprises administering during a defined time period a gradually increasing amount of said short acting NO donor in an intermitting manner.

According to the invention, the term “NO donor” refers to either nitric oxide itself or to any molecule which is capable of releasing nitric oxide (NO) after having been administered to a subject.

The term “short acting NO donor” refers to a NO donor with a halftime of less than 60, preferably with a halftime of less than 45, more preferably with a halftime of less than 30 and most preferably with a halftime of 15 minutes or less after having been administered to a subject.

It is to be understood that, in the context of the present invention, the term “halftime” refers to the half-life and/or to the half-life time of the NO donor in the subject's body, in particular in the subject's blood plasma.

Preferably, the short acting NO donor is selected from the group consisting of octyl nitrite, amyl nitrite, nitroglycerin, isosorbide dinitrate, isoididmononitrat, isoididinitrat and mannitol hexanitrate.

Preferably, the NO donor of the invention is not selected from the group consisting of isosorbide mononitrate, pentaerythritol tetranitrate (PETN), diethylenetriamine NONOate (DETA-NONOate), molsidomine.

Preferably, the short acting NO donor is nitroglycerin.

In the case of nitroglycerin, the halftime and its persistence in the body of the subject has been intensively studied, see e.g. Armstrong et al. Circulation 59:585-588 (1979) or Armstrong et al. Circulation 62:160-166 (1980). In general, the halftime of nitroglycerin is 2 to 5 minutes.

According to the invention, the term “administration of an NO donor” means that a given dosage of the NO donor is administered. Depending on the way of administration, the skilled person will appreciate that the administration may take some time. In a preferred embodiment, the NO donor is administered in form of a spray, sprayable or injectable solution, chewable capsule, inhalable gas, inhalable aerosol or powder, granules, powder or a tablet, preferably a sublingual, buccal or chewable tablet, which means that the administration may be completed within seconds. Modes of administration of the NO donor are further discussed below.

According to the invention, the short acting NO donor is administered in an intermitting manner.

In the context of the present invention, the term “intermitting manner” means that the NO donor is administered in a way that its plasma or tissue levels are only elevated in a short-term manner after the NO donor has been administered but then again decline. This can be achieved, for example, if the administration of the short acting NO donor is followed by a time period without administration and then the NO donor is again administered to the subject. Moreover, the administration of a short acting NO donor in an intermitting manner according to the present invention has the effect that it mimics the physiological situation of the organism as, for example, comparable to the endogenous release of NO upon physical training. In other words, the NO donor of the present invention acts as a biomimetic when administered to a subject in an intermitting manner.

In a preferred embodiment, the plasma or tissue levels of the NO donor are elevated for not more than 180, 120, or 60 minutes, or for not more than 50, 40, 30, 15, 10 or 5 minutes.

In the context of the present invention, the term “in an intermitting manner” is to be understood that the short acting NO donor is not administered continuously, for example, by means of long term intravenous infusion or with the help of an implanted pump which constantly delivers the NO donor to the subject.

In the context of the present invention, the term “in an intermitting manner” is to be understood in that the short acting NO donor is not administered in a way that it is released for a prolonged, sustained period of time into the subject. This means that the short acting NO donor is not administered in a way that a prolonged bioavailability of a therapeutically sufficient amount of the NO donor or NO, respectively, is generated in the subject resulting in a long duration of therapeutic action. Thus, in accordance to the present invention, the short acting NO donor is not administered in form of an extended-release preparation resulting inevitably in a prolonged NO donor release into the subject, such as, for example, by means of using a transdermal GTN patch with a controlled release of GTN from a reservoir or by means of swallowing an oral retard preparation with a slow and thus sustained release (e.g. oral ISDN retard).

If the short acting NO donor is formulated in a sustained-release formulation, the mode of administration has to ensure that the NO donor is not released for a prolonged period of time to the subject. For example, this might be achieved by an only short-term adhesion of sustained-release mucosa-adhesive tablet to the mucosa, e.g. a sustained-release mucosa-adhesive buccal tablet, resulting in a short-term release of the NO donor into the subject.

Therefore, in a preferred embodiment, the short acting NO donor is formulated in a way that it is released over a short period of time after administration. Thereby, the NO donor may be released immediately (i.e. immediate-release formulation) as well as after a delayed period of time (delayed-release formulation) after having been administered to the subject.

In a preferred embodiment, the short acting NO donor is formulated in an immediate-release formulation. Examples of immediate-release formulations include, but are not limited to, sublingually administered tablets, powder, granules, films, chewable capsules or solution in form of a spray. Other examples include conventional (i.e. non-coated, non-retard) tablets or capsules for oral administration.

Rather, this term also means that there is an interval between two administrations of the NO donor, and that the NO donor is given several times, e.g. at least 1, 2, 3, 4, 5, 6, 8, 9, 12 or 16 times a day. Equally preferred is that the NO donor is administered at least once a day, preferably up to 2 to 3 times a day, and more preferably up to 4 times as day.

Preferably, the NO donor is administered three times a day.

As to the schedule of administration, the skilled person will appreciate that there are many ways to achieve the intermitting administration of the invention. For example it is possible to administer the donor at least once a day or, alternatively, at least three times a week for reducing the side effects.

Preferably, during the defined time period in which the amount of the NO donor is gradually increased, the NO donor may be administered at least once a day, more preferably twice, three times or four times a day, even more preferably under the proviso that the time period between two administrations of the NO donor is at least 1 hour, 2 hours or 4 hours, preferably at least 8 hours, more preferably at least 10 hours to 12 hours.

Although possible, it is not necessary that the time periods between two administrations of the NO donor are the same. Rather, it is preferred that these time periods differ, depending on the individual administration schedule.

In a preferred embodiment, the NO donor is administered at least on three days a week. However, the NO donor may also be administered on 4, 5, 6 or 7 days a week. In an especially preferred embodiment, the NO donor is administered at least on 3 or 4 days a week. In equally preferred embodiment, the NO donor is administered on 7 days a week.

Consequently, it is also envisaged in the context of the present invention that the short acting NO donor is administered several times a day, but not on all days of the week, preferably on every other day.

According to the invention, the short acting NO donor is administered during a defined time period in a gradually increasing amount. In the context of the present invention, it is to be understood that the term “defined time period” may vary dependent on the particular side effect to be reduced and, in addition, dependent on the patient to be treated.

In the context of the present invention, the defined time period may last for at least 3 days to 40 weeks, preferably for 1 week to 16 weeks, more preferably for 3 to 4 weeks, and most preferred for at least two weeks.

Preferably, the defined time period lasts for two weeks.

Equally preferred is that the defined time period lasts for 3 days to one week.

Also preferred is that the defined time period lasts for 3 weeks.

It is to be understood that the defined time period may also last for 2 to 3 weeks or that any other lengths may be possible to define the time period according to the present invention in which an gradually increasing amount of an NO donor of interest is administered to the subject in need thereof for reducing the side effects which go along with a treatment with said NO donor.

The length of the time period in which the amount of the NO donor is gradually increased as well as the individual administration schedule to prevent side effects may also be adjusted individually for each patient. In the context of short acting NO donators, for example, it is known that the level and duration of side effects strongly correlate with and/or depend on the individual sensitivity of the patient, on its bodyweight, physical condition, blood pressure etc.

Preferably, said defined time period is the initial phase of the treatment with the short acting NO donor.

In a preferred embodiment, the initial phase lasts for two weeks.

In another preferred embodiment, the initial phase lasts for three or four weeks.

In yet another preferred embodiment, the initial phase lasts for three weeks.

Side effects of the treatment with a short acting NO donor are similar to the side effects of long acting NO donors, well-known in the art and include headache, dizziness, lightheadedness, syncope, vertigo, drowsiness, severe and prolonged headache, cerebral ischemia, faintness and somnolence.

Hence, in a preferred embodiment, the side effect to be reduced is selected from the group consisting of headache, dizziness, lightheadedness, syncope, vertigo, drowsiness, cerebral ischemia, faintness and somnolence.

More preferably, the side effect to be reduced is selected from the group consisting of headache, dizziness, lightheadedness, vertigo and drowsiness.

Most preferably, the side effect to be reduced is headache.

As the skilled person will appreciate, the administration of the NO donor according to the present invention may include an administration in one or more dosage forms, e.g. tablets or hubs (puffs). That is, the NO donor may be provided and administered in form of a spray or in form of a sublingual powder, preferably provided in sachets. For example, one administration may include the administration of two tablets or one to three hubs (puffs) or one to three sachets of sublingual powder, or any combination thereof.

Accordingly, in a preferred embodiment, the short acting NO donor is administered in form of a powder, granules, a tablet, a capsule, a liquid, a gel, a solution or an aerosol.

Preferably, the NO donor is administered in form of a powder.

More preferably, the NO donor is administered in form of a powder, wherein the powder is in form of a sublingual powder. Even more preferably, the sublingual powder is provided in form of sachets or, alternatively, in form of stick packs.

According to the present invention, the initial phase is the phase in which dosages are administered in increasing amounts towards a predefined dosage (forced titration) or towards a dosage for optimal clinical treatment (individual titration). The initial phase should be as short as possible to reach the therapeutic dosage rapidly but long enough to prevent side effects and to allow sufficient patient adjustment. In the context of short acting NO donators, for example, it is known that the level and duration of side effects strongly correlate with and/or depend on the individual sensitivity of the patient, on its bodyweight, physical condition, etc. Therefore the optimal titration schedule could also be analyzed and monitored by a medical doctor or another skilled person.

However, it is also possible that the treatment with the NO donor has already started and has been stopped, e.g. due to the occurrence of side effects.

Hence, in another preferred embodiment, the defined time period is a phase which takes place during treatment with the NO donor, preferably after a first dosage of the NO donor has been administered which results in side effects.

In this case, the patient's treatment is restarted on the highest dose that is tolerated without side effects.

After a predefined or patient-optimized dosage is reached, the dosage is kept stable.

During this defined time period, according to the invention, the amount of the short acting NO donor administered is gradually increased. Said increase may occur stepwise, i.e. in that the amount is increase once per week, followed by several administrations of the same amount. Alternatively, said increase may occur stepwise in that the amount is increased on a daily basis or after each administration until a final, a maximum concentration (or a plateau concentration) or therapeutic effective dose is reached. In a preferred embodiment, the initial amount of the short acting NO donor which is administered during the defined time period is a subtherapeutical or less effective amount.

In the context of the present invention, the term “gradually increased” or “gradually increasing amount” means that the dosage which is administered to the patient is increased at least two times before the final and effective dosage for the treatment is administered. This can be achieved by increasing the dose for example every day, every other day, every third day, every week, fortnightly, every month, every other month or any other suitable time schedule. As the aim of a treatment is to achieve an effective therapeutic dosage as soon as possible and the aim of the intervention is the reduction of side effects, the skilled person would start with the highest dosage that is tolerated by the patient without side effects. The interval for the initial period may vary due to the nature of the side effects and to the constitution of the patient.

Preferably, a suitable initial dosage of nitroglycerin (GTN) for sensitive patients may be 0.15 mg of GTN with a biweekly increase of 0.15 mg of GTN, with the aim to reach a final and effective dosage of GTN of 0.45 mg which is then administered for, e.g., a time period of 16 weeks. The initial dose will be chosen to be lower if the patient complains about side effects.

Equally preferred is that the administration starts with a dosage of 0.15 mg of GTN, characterized in that this dosage is administered for a time period of two weeks. After the starting dosage has been administered for two week, the dosage is then, in a next step, increased to an amount of 0.3 mg of GTN. GTN is then administered for the next two weeks in an amount of 0.3 mg. In a second step, the dosage is increased to a final and effective dosage of 0.45 mg of GTN. In this example, the total treatment period lasts for 20 weeks, encompassing an initial period in which the dosage is gradually increased in two steps, starting with a dose of 0.15 mg of GTN for a time period of 2 weeks, followed by a dose of 0.3 mg for another 2 weeks and finally followed by a 16 week long treatment with the effective therapeutic dose of 0.45 mg of GTN.

In another preferred embodiment, the initial dosage may an amount of 0.3 mg of GTN with the dosage increasing gradually in two steps over a defined time period of three weeks to reach the optimal effective therapeutic dosage of 0.7 mg of GTN. The dosage is increased by 0.2 mg of GTN in every step. Every dosage is administered for at least one week, with the final therapeutic dose administered for at least one week, e.g. for a time period of two weeks, four weeks, ten weeks, 3 months, 6 months or any another suitable time interval.

Preferably, the drug administration starts with an initial dose of 0.3 mg nitroglycerin for one week, followed by a dose of 0.5 mg nitroglycerin for another week (titration step 1).

More preferably, the drug administration continues from week three on with a dose of 0.7 mg nitroglycerin (titration step 2) administered as a therapeutic dose for another ten weeks. Even more preferably, in this context, the nitroglycerin is administered to the patient at least three times a day.

In yet another preferred embodiment, the initial dosage may be 0.4 mg of GTN with a dosage increasing gradually in three steps over a defined time period of four weeks to reach the final and effective dosage of 1.2 mg of GTN. The starting dosage of 0.4 mg GTN could be administered for one week, the next dosage of 0.8 mg of GTN would then be administered for another (i.e. the second) week, the next dosage of 1.0 mg of GTN would be administered for another (i.e. the third) week. After three steps, the effective therapeutic active dosage of 1.2 mg GTN is finally reached in the fourth week.

Equally preferred is that the initial dosage of GTN may be administered in an amount of less than 0.15 mg of GTN, preferably in an amount which is in a range of 0.05 to 0.1 mg of GTN.

In yet another preferred embodiment, the starting dosage of another NO donor, such as for example ISDN, could be 1.25 mg. The dosage is, for example, increased gradually in the initial time period of three weeks from the starting dosage in two steps to reach the final therapeutic dosage of 5 mg.

Equally preferred is that the initial dosage of ISDN may be administered in an amount of less than 1.25 mg of ISDN, preferably in an amount which is in a range of 0.5 to 1 mg of ISDN.

In yet another preferred embodiment, the initial dosage of ISDN may be administered in an amount higher than 1.25 mg of ISDN, preferably in an amount which is in a range of 1.5 to 3 mg of ISDN.

As mentioned above, the time period in which the starting dosage is gradually increased in at least two steps towards the therapeutically effective final dosage can vary significantly and also strongly depends on the individual response of the patient that is treated.

If the titration consists of at least two steps and the titration steps are relatively big, which means the following dosage is 100%, 130%, 140%, 150%, 200%, 250%, 300% or 400% of the previous, the duration of the individual titration step may be prolonged to allow optimal adjustment to the individual patient's response.

In one preferred embodiment of the invention, the defined time period is an ultra-short with a length of three days with a stepwise increase every day. Equally preferred is a time period of eight days with a stepwise increase every other day. Another preferred embodiment is in form of a time period of 12 days with a stepwise increase of dosage every third day. Yet another preferred embodiment is a time period of 3 weeks with a stepwise increase every week. Yet another preferred embodiment is a time period of six weeks with a stepwise increase of dosage every other week. If the patient is more sensible to the side effects yet another preferred embodiment of the invention is a long initial phase with a length of 9 weeks and a stepwise increase every third week.

Yet another preferred embodiment is a time period of 12 weeks with a stepwise increase every third to fourth week.

The length of the total titration period as well as the titration schedule may be varied due to the NO donor, the patient sensitivity and the individual patient's response.

The stepwise increase can be predefined in form of a fixed schedule (forced titration) or the increase can be triggered by a physiological response.

Preferably, at the onset of said defined time period, the short acting NO donor is administered in a subtherapeutical or less effective amount. According to the invention, the term “subtherapeutical amount” refers to an amount which is not therapeutically effective.

According to the invention, a “less effective amount” represents an amount of the short acting NO donor which is already therapeutically active, but wherein the administered amount is not sufficient for triggering the desired therapeutic effect.

According to the invention, a “therapeutically active amount” is defined as an amount which results in the desired therapeutic effect, such as, for example, the intended physiological response.

Subtherapeutical as well as therapeutically active amounts of NO donors are known in the art. For example, with respect to nitroglycerin (GTN), the initial (and thereby subtherapeutical) amount may be in the concentration range of 0.05 mg, 0.1 mg, 0.2 mg or 0.3 mg, but are not limited thereto.

This amount may then be stepwise increased, e.g. within four weeks or within any of the periods as defined above, to 0.5, 0.6, 0.7, 0.8, 1.2 or 2.0 mg.

Comparable amounts are also known for other short acting NO donors.

The amount of the NO donor may be also stepwise increased from 0.05 to 10 mg, or, alternatively, from 0.1 to 100 mg, dependent on the nature of the NO donor.

In another example, the initial subtherapeutical or less effective amounts of ISDN may be in the concentration range of, but are not limited to, 0.25 mg, 0.5 mg, 0.6 mg or 0.7 mg. This amount may then be stepwise increased, i.e., within three weeks towards therapeutically active amounts in the concentration range of 1.25, 2.5, 3.75, 5, 10, 15, 20 mg or any combination thereof.

Preferably, the stepwise increase of the initially administered subtherapeutical (or less effective) amounts towards the therapeutically active concentration ranges of 1.25 mg to 5 mg (or more) may be obtained by an administration of ISDN in form of several independent puffs, or, alternatively, by the administration of one or more sublingual tablet(s), or any combination thereof.

In a preferred embodiment of the invention, the final amount of said short acting NO donor administered at the end of the defined time period is a therapeutically active amount.

The NO donor can be administered in any suitable way so that it can be incorporated into the subject. This includes a parenteral or an intravenous administration as well as the administration to a mucous membrane of the subject.

Consequently, in a preferred embodiment of the present invention, the short acting NO donor is administered lingually, sublingually, inhalatively, buccally, transmucosally or oromucosally.

Preferably, the NO donor is not swallowed.

Most preferably, the NO donor is administered parenterally.

The NO donor can be formulated in any suitable way for the above mentioned administration modes.

In case of a lingual, sublingual or oromucosal administration, it is preferred that the short acting NO donor, preferably nitroglycerin or ISDN, is administered with the help of a spray, sprayable solution, a chewable capsule or in the form of a tablet, preferably a sublingual, buccal or chewable tablet, powder or granules or even by an inhalator device, from which the NO donor can be easily inhaled and adsorbed. It is equally preferred that the NO donor is administered in the form of an inhalable gas, aerosol or powder. Corresponding formulations are known to the person skilled in the art and include the formulation in a liquid in suitable buffers, in a gas, aerosol, as tablets, powder or granules.

Preferably, the administration of the NO donor is a non-topical administration, i.e., that the NO donor is not administered to the skin of the subject. In the context of the present invention, the term “skin” excludes mucous membranes of the subject.

Especially, the term “intermitting manner” means that the short acting NO donor is formulated in a manner allowing an administration in an intermitting manner. The NO donor is formulated in a way that allows a fast release of the NO donor from the formulation. Thereby, the NO donor may be released immediately (i.e. immediate-release formulation) as well as after a delayed period of time (delayed-release formulation) out of the preparation (e.g. formulation or dosage form) after having been administered to the subject. This includes e.g. formulations which do not hold back the NO donor for a longer time period and which are not characterized by a slow, sustained release, e.g. extended-release or retard preparations, but which release the NO donor within e.g., 30 or 15, 10, 5 minutes or 1 minute. The release may be also triggered upon an external signal, such as UV-light, temperature, ultrasound or a mechanical trigger.

According to a preferred embodiment, the short acting NO donor is used for the treatment or prevention of an arterial insufficiency.

According to the invention, the term “arterial insufficiency” refers to any insufficient blood or oxygen supply or any other insufficient supply of a tissue which is provided by an artery. This insufficient supply can be overcome by the methods and uses of the present invention wherein an NO donor is used to increase the supply of a given tissue. The arterial insufficiency may occur both during physical rest and during an exercise.

In a preferred embodiment of the present invention, the arterial insufficiency is due to insufficient oxygen or blood supply of a tissue supplied by the artery or a bypass or shunt during physical rest or exercise.

According to a further preferred embodiment, the arterial insufficiency is due to an increased demand of oxygen or blood flow of a tissue supplied by the artery or a bypass or shunt.

This increased demand of oxygen or blood flow can have several reasons including but not limited to increased sport or physical activity, and increased mental activity or a disease requiring an increased demand of oxygen or blood flow.

According to a further preferred embodiment, the arterial insufficiency is characterized by a partial (stenosis) or complete occlusion of an arterial vessel. In the context of the present invention, the term “partial occlusion” is equivalent to a stenosis.

The partial or complete occlusion of an arterial vessel is a well-known phenomenon. It can have various reasons including, but not limited to, deposition of material in the blood vessels (including non-revascularisable stenoses), compression from external tissue or fluid next to the vessel (including disturbance in diastolic myocardial relaxation), vascular spasm, dysfunction of the endothelium of the vessel resulting in a paradoxic vasoconstriction during exercise or microvascular impairment due to endothelial dysfunction or smooth muscle cell abnormalities.

In a preferred embodiment, the arterial insufficiency is due to the deposition of material in the blood vessels.

The deposition of materials in the blood vessels is a well-known phenomenon resulting e.g. in atherosclerosis.

In a further preferred embodiment, the arterial insufficiency is due to an external or internal compression of an artery.

An internal compression of an artery may be due to an edema but also to a tumor putting pressure on the artery. Furthermore, this includes a vasospastical constriction of the artery as e.g. in Prinzmetal's angina. In addition, this also includes the paradoxic vasoconstriction which e.g. sometimes occur in an endothelial dysfunction or constricted small arterial vessels due to endothelial or smooth muscle cell dysfunction.

An external compression may be due to an accident or any external force which can put pressure on an artery.

In a further preferred embodiment, the arterial insufficiency is a vascular disease.

According to a further preferred embodiment, the arterial insufficiency is a disease selected from the group consisting of atherosclerosis, an ischemic disease and a further chronic arterial disease.

In a further preferred embodiment, the arterial insufficiency is a coronary arterial insufficiency.

In a preferred embodiment, the coronary insufficiency is an atherosclerotic coronary arterial insufficiency, in particular coronary artery disease (coronary heart disease or ischemic heart disease), stable angina pectoris, unstable angina pectoris, myocardial ischemia or chronic myocardial ischemia, acute coronary syndrome, or myocardial infarct (heart attack or ischemic myocardial infarct).

In a further preferred embodiment, the coronary insufficiency is a non-atherosclerotic, in particular coronary microvascular disease or small vessel disease, Prinzmetal's angina and cardiac syndrome X.

In a further preferred embodiment, the arterial insufficiency is a cerebral arterial insufficiency (intra- or extracranial).

In a preferred embodiment, the cerebral arterial insufficiency is an atherosclerotic cerebral arterial insufficiency, in particular cerebral ischemia, extracranial carotid artery disease, extracranial vertebral artery disease, pre-stroke, transient ischemic attack (mini stroke), stroke, vascular dementia, ischemic brain disease, or ischemic cerebrovascular disease.

The cerebral arterial insufficiency may also be ischemic microvascular brain disease, small vessel vascular dementia, subcortical arteriosclerotic encephalopathy (Binswanger's disease), Alzheimer's disease, or Parkinson's disease.

In a preferred embodiment, the arterial insufficiency is a peripheral arterial insufficiency.

In a preferred embodiment, the peripheral arterial insufficiency is an atherosclerotic peripheral arterial insufficiency, in particular peripheral vascular disease (peripheral artery disease (PAD) or peripheral artery occlusive disease (PAOD), including lower and upper extremity arterial disease).

In a preferred embodiment, the peripheral arterial insufficiency is an non-atherosclerotic peripheral arterial insufficiency, in particular Raynaud's syndrome (vasospasmatic), thrombangiitis obliterans, endangitis obliterans or Buerger's disease (recurring progressive inflammation and thrombosis (clotting) of small and medium arteries and veins of the hands and feet), vascular inflammatory disease (vasculitis), diabetic ischemia, diabetic neuropathy and compartment syndromes.

In a further preferred embodiment, the arterial insufficiency may be an intestinal arterial insufficiency, in particular an atherosclerotic intestinal arterial insufficiency, in particular ischemic bowel disease, mesenteric ischemia, or mesenteric infarction.

In a further preferred embodiment, the arterial insufficiency may be an urogenital arterial insufficiency, in particular an atherosclerotic urogenital arterial insufficiency, in particular erectile dysfunction, renal artery disease, renal ischemia, or renal infarction.

In a further preferred embodiment, the arterial insufficiency may be a nerval arterial insufficiency, in particular tinnitus.

Furthermore, the arterial insufficiency may be in the context of scleroderma (systemic sclerosis).

Furthermore, the arterial insufficiency may be in the context of fibromuscular dysplasia.

In a preferred embodiment, the arterial insufficiency is a central retinal artery insufficiency, in particular an atherosclerotic central retinal artery insufficiency, in particular ocular arterial insufficiency.

In a further preferred embodiment, the arterial insufficiency is characterized by an absence of an endothelial dysfunction.

The endothelial dysfunction is a well-known systemic pathological state of the endothelium and can be broadly defined as an imbalance between vasodilating and vasoconstricting substances produced by or acting on the endothelium.

In a further preferred embodiment, the arterial insufficiency is a chronic arterial insufficiency. In the context of the present invention, the term “chronic arterial insufficiency” means that the course of the arterial insufficiency is chronical and often progredient.

According to a further preferred embodiment, the chronic arterial insufficiency includes endothelial dysfunction, atherosclerosis, coronary artery disease (coronary heart disease or ischemic heart disease), stable angina pectoris, coronary microvascular disease or small vessel disease, Prinzmetal's angina and cardiac syndrome X, vascular dementia, ischemic brain disease, or ischemic cerebrovascular disease, ischemic microvascular brain disease, small vessel vascular dementia, subcortical atherosclerotic encephalopathy (Binswanger's disease), Alzheimer's disease, Parkinson's disease, peripheral vascular disease (peripheral artery disease (PAD) or peripheral artery occlusive disease (PAOD), thrombangiitis obliterans, endangitis obliterans or Buerger's disease, vascular inflammatory disease (vasculitis), fibromuscular dysplasia, diabetic ischemia, diabetic neuropathy, ischemic bowel disease, erectile dysfunction, renal artery disease, tinnitus, and scleroderma (systemic sclerosis).

In a further preferred embodiment, in case that the short acting NO donor is used for the treatment or prevention of an arterial insufficiency, the short acting NO donor is administered in a final amount effective for the induction of arteriogenesis.

This implies that the short acting NO donor can also be administered at various time points or, alternatively, for various time periods where there is no need for vasodilation and the relief of symptoms such as angina pectoris.

The skilled person will appreciate that this amount will depend on the patient in need of the treatment to which the short acting NO donor is administered. Generally, the amount to be administered may be between 0.1 mg and 40 mg per day, but this can vary due to the weight of the subject, its hemodynamic response to the short acting NO donor, the nature of the short acting donor itself and/or the severity of the disease. That is, if the short acting NO donor is in form of nitroglycerin (GTN), the daily amount to be administered to a patient in need thereof may be in the range of between 0.1 mg and 10 mg of GTN, preferably in the range of between 0.5 mg and 5.0 mg of GTN. Alternatively, if the short acting NO donor is in form of sublingual or non-retard oral isosorbide dinitrate (ISDN), the daily amount to be administered may be in the range of between 1 mg to 100 mg of ISDN, preferably in the range of between 4 mg to 20 mg of ISDN.

In a preferred embodiment, treatment with a short acting NO donor may be accompanied by physical exercise or by the application of an endogenous force to the arterial vessel.

According to the invention, the term “physical exercise” means any training of the subject, including but not limited to training in exercise rooms, jogging, walking (including walking on a tread mill), nordic walking, swimming, dancing, cycling and hiking. The skilled person will appreciate that any exercise will be helpful in the context of the invention, provided that it is performed in conjunction with the administration of the short acting NO donor.

In a further aspect, the present invention also relates to a short acting NO donor for use in a method for the prevention or treatment of an arterial insufficiency, wherein the NO donor is administered in an intermitting manner, and wherein during the initial phase of the administration, the amount of said short acting NO donor is gradually increased.

Equally, the present invention also relates to a method for the prevention or treatment of an arterial insufficiency, wherein a short acting NO donor is administered in an intermitting manner, and wherein during the initial phase of the administration, the amount of said short acting NO donor is gradually increased.

All embodiments defined above with respect to the first aspect of the invention and relating to the short acting NO donor, the method for the prevention or treatment of an arterial insufficiency, the administration in an intermitting manner, and the increase of the amount of said short acting NO donor at the initial phase of the administration also apply to this aspect of the invention.

According to the invention, as to the total time of the treatment or prevention, it is possible to administer the short acting NO donor for a period of several weeks or months.

In a preferred embodiment, the NO donor is administered for a time period of up to 8 to 10 weeks. It is equally preferred to administer the NO donor for 3 to 6, 3 to 8, 3 to 10, 4 to 8, 4 to 10, or 4 to 12 weeks. These time ranges are only examples and may vary depending on the individual schedule of the subject.

In a preferred embodiment, the NO donor is taken at least three times a week for at least 8 weeks, in particular for at least 12 weeks.

In another preferred embodiment, the NO donor is taken at least two to three times a day for 7 days a week and for at least 4 weeks, preferably for at least 8 weeks and more preferably for at least 12 weeks.

In a further preferred embodiment, the NO donor is taken not longer than 6, 8 or 12 months. However, it is also possible to take the NO donor for 2, 3 or even more years. Furthermore, it is also possible that the NO donor is administered for decades or even through the whole life of the subject.

In the context of such long term administrations, it is preferred that the NO donor is administered three or four times a week or at least three times a week.

The present invention also relates to a method of promoting collateral circulation comprising the step of exposing a subject to a therapeutically effective amount of an NO donor, wherein the therapeutically effective amount of the NO donor promotes arteriogenesis sufficient to augment collateral circulation in a physiological or pathological condition, and wherein during the initial phase of the administration, the amount of said short acting NO donor is increased.

The term collateral circulation describes the circulation of blood through so-called collateral vessels. These vessels are small arterioles, which are part of a network that interconnects perfusion territories of arterial branches. In the case that the main artery itself is not capable of sufficiently supplying a tissue, e.g. due to an arterial occlusion, these collateral vessels are recruited and can develop to large conductance arteries, to bypass the site of an arterial occlusion and/or to compensate blood flow to ischemic territories supplied by the or insufficient artery. In the context of the present invention, the promotion of collateral circulation occurs via arteriogenesis.

According to the invention, the term “physiological condition” denotes any condition of the subject which is not related to any disease.

According to the invention, the term “pathological condition” denotes any condition of the subject which is related to a disease.

Preferably, the subject suffers from an arterial insufficiency.

All features and preferred embodiments discussed above for the method of treating or preventing an arterial insufficiency also apply to the method of promoting collateral circulation.

With respect to the aspects defined above where the NO donor is administered in a manner sufficient to induce arteriogenesis this manner is preferably an intermitting manner as defined above.

The invention is further explained by the following figures and examples, which are not intended to limit the present invention.

### FIGURE LEGENDS

FIG. 1: Schematic representation of the multi-centre, randomized, placebo-controlled, double-blind trial to assess the efficacy and safety of nitroglycerin sublingual powder on the walking distance in a scheduled forced titration design in patients with peripheral artery occlusive disease (PAOD) and intermittent claudication (EUDRA-CT 2016-004460-19) (Example 3).

FIG. 2: Interim analysis of headache incidence in a multi-centre placebo-controlled, double-blind trial to assess the efficacy and safety of nitroglycerin sublingual powder on the walking distance in a scheduled forced titration design in patients with peripheral artery occlusive disease (PAOD) and intermittent claudication after finalized 3 week titration and first week of regular treatment (Example 3).

### EXAMPLES

### Example 1

A 35-year old men who is known to show severe headache when taking sublingual nitroglycerin in a therapeutic dose was put on a less effective amount of approximately 0.15 mg GTN TID (morning, noon, evening) sublingual powder to start. At this dosage he had no headache or other known GTN side effects. After a week he increased the dose to 0.3 mg GTN, after the second week to 0.5 mg, after another week he reached the final dosage of 0.7 mg without side effects.

### Example 2

A 51-year old woman who responded with nausea and mild headache to an initial dose of 0.4 mg sublingual nitroglycerin was put on 0.15 mg TID (morning, noon, evening) to start. At this dosage she had no nausea or headache or other known GTN side effects. After a week, she increased the dose to 0.3 mg GTN TID, after the second week she reached the final dosage of 0.4 mg TID without side effects.

### Example 3

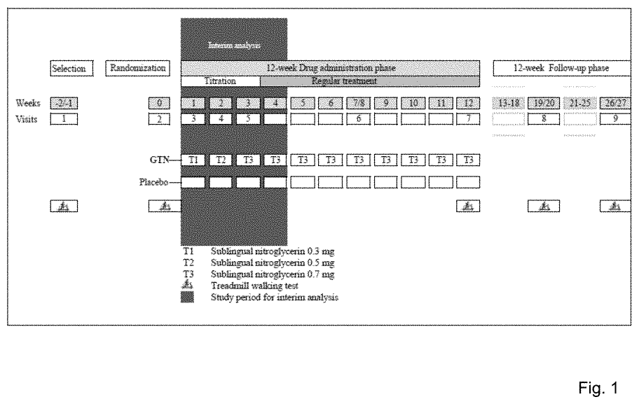

In a multi-centre, randomized, placebo-controlled, double-blind trial the efficacy and safety of nitroglycerin sublingual powder on the walking distance in a scheduled forced titration design in patients with peripheral arterial occlusive disease (PAOD) and intermittent claudication is assessed (EUDRA-CT 2016-004460-19). This clinical Phase IIa proof-of-concept study with 50 patients suffering from peripheral artery disease (PAD) and a pain free walking distance <200 metres (stadium Fontaine IIb) investigates the efficacy of an intermittent administration of nitroglycerin on the walking distance via the induction of arteriogenesis and consists of a 12-week phase of drug administration and a 12-week follow up phase without drug administration. In order to reduce the expected side effects of the short acting NO donor administration the study does not start with the therapeutic dose but follows a forced titration with two titration steps as described in the following. Drug administration starts with a low initial dose of 0.3 mg nitroglycerin for one week, followed by 0.5 mg (titration step 1) for another week. From the third week on the patients receive the final therapeutically active dosage of 0.7 mg nitroglycerin (titration step 2) for another ten weeks (FIG. 1). The patients are advised to take the dosages in the morning, noon and evening so that the short acting NO donor is administered in an intermittent manner.

Study parameters are pain free walking distance (ICD=initial claudication distance) and maximum walking distance (ACD=absolute claudication distance) assessed by treadmill walking tests. These parameters are commonly accepted parameters in studies investigating the efficacy of therapies regarding peripheral artery disease. These parameters are the primary and secondary endpoints. Therefore for both parameters the increase in walking distance is measured. For the primary endpoint the difference from the walking distances of treadmill tests performed before the start of the drug administration phase (baseline) to week 12 (at the end of drug administration phase) is calculated.

For the secondary endpoint the difference of the walking distances of treadmill test performed before the start of the drug administration phase (baseline) to 6 weeks after the drug administration phase (first follow up visit at week 19-20) and to the end of the 12-week follow up phase (second follow up visit at week 26) is measured.

Additional secondary parameters are ankle-brachial index (ABI, difference from baseline to week 12, to week 19-20 and to week 26), questionnaires EQ-5D and ICQ (difference from baseline to week 12 and 19-20) and Oxygen saturation (SpO₂ before start of drug administration phase, at the start of the drug administration phase, at the end of the 12-week drug administration phase, 6 weeks after drugs administration phase and at the end of the 12-week follow up phase).

Participants report adverse effects during the study with headache being one of the adverse effects.

### Interim Analysis of the Ongoing Study

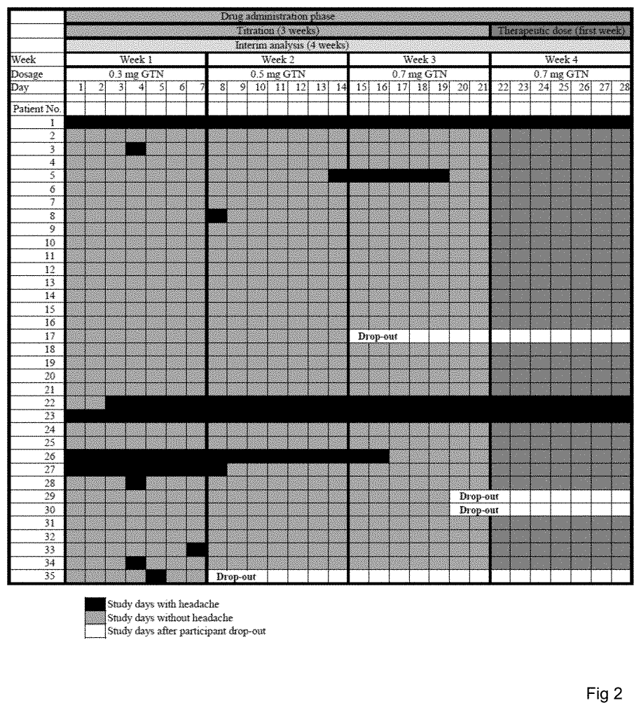

The study has not yet been finalized but an interim analysis regarding headache as an adverse effect of the study drug has been performed (FIG. 2, Table 1-3).

At the time point of the interim analysis the study was still blinded, so with regard to patients' reported adverse event headache it could not be distinguished between the frequency of headache in the verum and the placebo group.

As the titration of the study medication takes place in the first 3 weeks, the analysis is focused on reported headache in the first three weeks plus in week 4 to also monitor possible late or ongoing side effects.

The analysis includes 35 participants so far which are at different time points of the study protocol. In FIG. 1 the individual headache days of the 35 participants are shown. Some participants have already reached the end of the 12-week treatment period and are in the follow-up period, some participants are still on treatment, but all participants (33) have surpassed the titration phase and are on the final dosage of 0.7 mg GTN.

TABLE 1
Number of participants and the
number of weekly and total study days.

| | |
|---|---|
| Number of participants | 35 |
| Number of study days | 245 |
| per week | (35 × 7) |
| Number of study days | 980 |
| (week 1-4) | (35 × 7 × 4) |
| Number of study days total | 2940 |
| (drug administration phase) | (35 × 7 × 12) |
| Number of drop-out days | 487 |
| (drug administration phase) | |
| and not completed days of | |
| patients on treatment | |
| Number of study days | 2453 |
| corrected by drop-out days | |
| (drug administration phase) | |
| and not completed days of | |
| patients on treatment | |
| Number of drop-out days | 53 |
| (week 1-4 drug | |
| administration phase) | |
| Number of study days | 927 |
| corrected by drop-out days | |
| (week 1-4 drug | |
| administration phase) | |

TABLE 2
Number and percentage of participants with and
without headache in the first 4 weeks on a weekly basis.

| Week | 1 | 2 | 3 | 4 |
|---|---|---|---|---|
| Dosage (GTN) | 0.3 mg | 0.5 mg | 0.7 mg | 0.7 mg |
| Participants with | 10 | 7 | 5 | 3 |
| headache (n) | | | | |
| Headache (%) | 28.6 | 20.0 | 14.3 | 8.6 |
| Study days (n) | 245 | 245 | 245 | 245 |
| Drop-out days (n) | 0 | 7 | 18 | 28 |
| Study days corrected by | 245 | 238 | 227 | 217 |
| drop-out days (n) | | | | |
| Headache days (n) | 38 | 31 | 29 | 21 |
| Headache days (%) | 15.5 | 13.0 | 12.3 | 9.7 |

The number of participants with headache is subsequently reduced from n=10 in week 1 over n=7 in week 2 to n=5 in week 3 and n=3 in week 4.

Referring to the 35 participants this represents 28.6% of the participants in week 1. This initial percentage is reduced subsequently to 20% in the second week, 14.3% in the third and 8.6% in the fourth week.

Of the 35 participants 6 participants had headache on a single day in the first 4 weeks, but not during the first three days. 6 different participants had headache that lasted more than one day, in 5 of these the headache lasted more than a week.

Of the 927 study days (number of study days corrected by drop-out days in week 1-4 of the drug administration phase) during the first 4 weeks of the trial the participants had headache on 118 days which is an overall percentage of 12.7% with 15.5% in week 1, 13.0% in week 2, 12.3% in week 3 and 9.7% in week 4.

245 participant headache days were recorded for a total of 2453 study days so far, which amounts to 10%.

209 headache days (85% of the total 245 headache days) were recorded for 3 participants that continuously had headache in the first 4 weeks and in the further course of the study.

There was one possibly headache related drop-out recorded during the titration in the first 4 weeks. In the further course of the therapy 2 additional participants dropped out due to headache.

### CONCLUSION

Due to the provisional character of the interim analysis it is not clear how an unblinding of the study data will influence the study outcome with regard to the side effect headache.

On basis of literature data an overall headache frequency of below 30% in the present study can be considered as low. During the therapy with short acting NO donors such as nitroglycerin or ISDN, headache is reported to be generally a very common side effect with a reported frequency of up to 64% when short acting NO donors are administered for therapeutical means.

In the present study it was found that the overall headache frequency started on a low 28.6% in the first week and was subsequently reduced during the titration period to 8.6% in week 4. This is a low frequency in comparison to the reported rate of headache in the literature for sublingual nitroglycerin.

The percentage of headache as well as the intensity of headache during the therapy with nitrates is known to be dose-dependent. Therefore higher doses are expected to trigger a higher number of side effects. Without titration a dosage of 0.7 mg GTN is to be expected to generate more side effects than a dosage of 0.3 mg GTN.

In contrast thereto the present study did not monitor an increased headache frequency in week 2, 3 or 4 as a result of the increased dosage. Therefore the study results may confirm that the titration regime prevents a dose related increase of headache as a side effect, although it could not be distinguished between the frequency of headache in the verum and the placebo group due to the blinded data.

The adjustment process under a titration regime is expected to need some days, so that headaches in the first days of the overall titration period or the first days after each titration step might to be expected more frequent than in the following days or weeks. The titration starting dose (0.3 mg GTN) in this trial is significantly lower than the final therapeutic dosage (0.7 mg GTN) to smoothen the adjustment process. There are only 5 participants (14.3%) that have headache during the first days of treatment which is considered a lower percentage than expected without titration and a start with the therapeutical dose of 0.7 mg GTN.

Limitations of the Interim Analysis and/or the Trial in Respect of the Side Effect Headache

It is known, that irrespective of the type of clinical study and investigated drugs headache is a common side effect due to the high frequency in which spontaneous headache occurs in the population. In a study covering a period of twelve weeks spontaneous headache is to be expected besides drug-induced side effects. So, single headache days in the course of the study may be spontaneous headache and not necessarily drug-induced.

Therefore, it is anticipated that beside GTN-induced headache also spontaneous headache will be reported during the study in the verum as well as in the placebo group. This will probably influence the final number of drug-induced headaches.

In the present trial a history of chronic headache or migraine was no exclusion criterion. So there might be a fraction of the trial participants with a history of chronic headache or migraine. Chronic headache or migraine may have led to an overestimation of headache frequency as three participants had continuous headache during the complete study (resp. until drop-out).

3 participants with possible chronic headache or migraine background account for 65% of the headache days in the first 4 weeks (82 out of 127 headache days) and 85% of the headache days (212 out of 251 headache days) in the overall study.

TABLE 3
Results with participants with possible chronic
headache or migraine background excluded.

| Week | 1 | 2 | 3 | 4 |
|---|---|---|---|---|
| Participants with Headache | 10 | 7 | 5 | 3 |
| (n) | | | | |
| Participants with possible | 3 | 3 | 3 | 3 |
| chronic headache or | | | | |
| migraine history | | | | |
| Participants with headache | 7 | 4 | 2 | 0 |
| corrected by participants with | | | | |
| possible chronic headache or | | | | |
| migraine history (n) | | | | |
| Headache corrected by | 20 | 11.4 | 5.7 | 0 |
| participants with possible | | | | |
| chronic headache or | | | | |
| migraine history (%) | | | | |
| Participants headache days | 19 | 10 | 7 | 0 |
| corrected by participants with | | | | |
| possible chronic headache or | | | | |
| migraine history (n) | | | | |
| Participant headache days | 7.8 | 4.2 | 3.1 | 0 |
| corrected by participants with | | | | |
| possible chronic headache or | | | | |
| migraine history (%) | | | | |

When excluding the 3 trial participants due to their possible background of chronic headache or migraine from the data analysis the percentage of participants (and participant days) with headache is even lower. The remaining participants with headache would then amount to only 15% of participant headache days and only 20% of participants with headache in the first week. Titration reduces them to 11.4% in week 2 and 5.7% in week 3. In week 4 no participant (0%) (except the 3 suspected chronic headache or migraine participants) complained of headache as a side effect. This trend continued in weeks 5-12.

So far there has only been one participant drop-out likely due to headache in the titration phase, in 2 later drop-outs headache may have been the underlying cause. These dropped out participants may have a potential chronic headache or migraine background.

### Expected Final Study Results

The study is planned to be finalized in 2019.

After the study is finalised and the data have been unblinded, we expect to find a lower frequency of headache in the titration verum group than reported for untitrated short acting nitrates use in literature data.

We expect the titration to reduce the side effect headache within the first three weeks to a low level or even prevent them completely despite the increasing dosage of nitroglycerin.

We also expect a low number of drop-outs during the beginning of the trial therapy, which is normally the most critical time in a trial as well as in real life therapeutic regimen.

1. A method of reducing side effects of a treatment with a short acting NO donor, comprising administering the short acting NO donor during a defined time period, wherein a gradually increasing amount of said short acting NO donor is administered in an intermitting manner.

2. The method according to claim 1, wherein the defined time period is an initial phase of the treatment with the short acting NO donor.

3. The method according to claim 1, wherein said defined time period is 3 days to 40 weeks long, preferably 1 week to 16 weeks long, more preferably 3 to 4 weeks long, and most preferred at least two weeks long.

4. The method according to claim 1, wherein an initial amount of the short acting NO donor administered during the defined time period is a subtherapeutical or less effective amount.

5. The method according to claim 1, wherein a final amount of the short acting NO donor administered during the defined time period is a therapeutically active amount.

6. The method according to claim 1, wherein the NO donor is administered at least once a day, preferably up to 2 to 3 times a day, more preferably up to 4 times a day.

7. The method according to claim 1, wherein the NO donor is administered lingually, sublingually, inhalatively, buccally, transmucosally or oromucosally.

8. The method according to claim 1, wherein said treatment is for the treatment or prevention of an arterial insufficiency.

9. The method according to claim 8, wherein the short acting NO donor is administered in a final amount effective for the induction of arteriogenesis.

10. The method according to claim 1, wherein the short-acting NO donor is selected from octyl nitrite, amyl nitrite, nitroglycerin, isosorbide dinitrate, isoididmononitrat, isoididdinitrat, mannitol hexanitrate.

11. The method according to any claim 1, wherein the short acting NO donor is administered in form of a powder, granules, a tablet, a capsule, a liquid, a gel, a solution or an aerosol.

12. The method according to claim 11, wherein the NO donor is administered by means of a dosage form delivering a uniform dose, preferably in form of a stick pack, a tablet, a capsule, a (pre-)metered spray, a (pre-) metered inhaler, a (pre-)metered delivery device, or, alternatively, by means of a dosage form delivering an adjustable dose, preferably in form of an adjustable metering device.

13. A short acting NO donor for use in a method for the prevention or treatment of an arterial insufficiency, wherein a short acting NO donor is administered in an intermitting manner, and wherein during an initial phase of the administration, the amount of said short acting NO donor is gradually increased.

14. The method according to claim 13, wherein the initial phase is 3 days to 40 weeks long, preferably 1 week to 16 weeks long, more preferably 3 to 4 weeks long, and most preferred at least two weeks long.

15. The method according to claim 13, wherein an initial amount of the short acting NO donor is administered during the initial phase of the administration, wherein donor this initial amount is a subtherapeutical or less effective amount.

16. The method according to claim 13, wherein a final amount of the administered short acting NO donor is a therapeutically active amount.

17. The method according to claim 13, wherein the short acting NO donor is administered at least once a day, preferably up to 2 to 3 times a day, more preferably up to 4 times a day.

18. The method according to claim 13, wherein the short acting NO donor is administered lingually, sublingually, inhalatively, buccally, transmucosally or oromucosally.

19. The method according to claim 13, wherein the short acting NO donor is selected from octyl nitrite, amyl nitrite, nitroglycerin, isosorbide dinitrate, isoididmononitrat, isoididdinitrat, mannitol hexanitrate.

20. The method according to claim 13, wherein the short acting NO donor is administered by means of a dosage form delivering a uniform dose, preferably in form of a stick pack, a tablet, a capsule, a (pre-)metered spray, a (pre-)metered inhaler, a (pre-) metered delivery device, or, alternatively, by means of a dosage form delivering an adjustable dose, preferably in form of an adjustable metering device.
